(19) United States
(12) Patent Application Publication
(10) Pub. No.: US 2019/0175491 A1
(43) Pub. Date: Jun. 13, 2019

(54) TOPICAL COMPOSITIONS OF APREMILAST

(71) Applicant: TORRENT PHARMACEUTICALS LIMITED, Gujarat (IN)

(72) Inventors: Jaya Abraham, Gujarat (IN); Vivek Mishra, Gujarat (IN); Kiran Chaudhari, Gujarat (IN); Vipul Mittal, Gujarat (IN)

(21) Appl. No.: 16/310,099

(22) PCT Filed: Jun. 14, 2017
(86) PCT No.: PCT/IB2017/053526

§ 371 Date: Dec. 14, 2018

(30) Foreign Application Priority Data
Jun. 15, 2016 (IN) 201621020502

(51) Int. Cl. A61K 9/00; A61K 31/4035; A61K 47/22; A61K 47/10; A61K 9/12; A61K 9/06; A61P 37/08

(52) U.S. Cl. CPC A61K 9/0014; A61K 31/4035; A61K 47/22; A61P 37/08; A61K 9/122; A61K 9/06; A61K 47/10

(57) ABSTRACT

The present invention relates to topical pharmaceutical compositions comprising Apremilast in an amount of about 0.1 to 5% w/w of the total composition and one or more pharmaceutically acceptable excipients and process for their preparation. The present invention further relates to method for treatment of skin diseases using topical pharmaceutical compositions comprising Apremilast.

### FIELD OF THE INVENTION

The present invention relates to topical pharmaceutical compositions comprising Apremilast and one or more pharmaceutically acceptable excipients and process for their preparation. The present invention further relates to method for treatment of skin diseases using topical pharmaceutical compositions comprising Apremilast.

### BACKGROUND

Apremilast is chemically known as N-{2-[(1S)-1-(3-Ethoxy-4-methoxyphenyl)-2-(methyl sulfonyl) ethyl]-1,3-dioxo-2,3-dihydro-1H-isoindol-4-yl} acetamide which can be characterized by the following chemical formula:

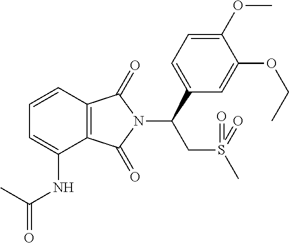 [SMILES: CCOc1cc([C@@H](CS(C)(=O)=O)N2C(=O)c3cccc(NC(C)=O)c3C2=O)ccc1OC]

Apremilast, approved as Otezla® in US and Europe, is a selective phosphodiesterase 4 (PDE4) inhibitor and increases intracellular cyclic adenosine monophosphate (cAMP) which decreases the expression of inflammatory cytokines such as tumour necrosis factor and interleukin-23 (IL-23). It is indicated for the treatment of active psoriatic arthritis and plaque psoriasis.

U.S. Pat. No. 6,020,358 discloses the compound Apremilast and process for its preparation. Several prior art patents disclose oral pharmaceutical compositions of Apremilast which includes U.S. Pat. No. 7,427,638 (tablets, capsule and aerosol dosage form of s-enantiomer of Apremilast), U.S. Pat. No. 9,351,957 (Amorphous solid dispersion of Apremilast), US application no. 20130164376 (Apremilast tablets composition), US patent application no. 20150306226 (Immediate release formulation of Apremilast by melt extrusion technique).

Apremilast is commercially available as tablets of 10, 20 and 30 mg for oral administration. Diarrhea, nausea and headache are most commonly reported adverse reactions with the use of oral Apremilast. Besides these, other gastrointestinal disorders are also observed with oral Apremilast.

Despite several options available, there remains an unmet need to develop a most suitable pharmaceutical composition of Apremilast. In particular, any new topical treatment that reduces the dosage and/or frequency of administration of Apremilast currently being used, or is capable of making a currently used treatment more effective, particularly with reduces GI related or systemic side effects, is constantly being sought.

PCT publication no. 2016198469 discloses topical formulations of water-free emulsions for penetration of alpha-hydroxy acids. Such topical formulations additionally comprise a pharmaceutically active medicinal product. Another PCT publication no. 2016198472 discloses topical delivery system comprising of a water-free emulsion of a discontinuous polar phase in a continuous lipid phase that will deliver certain compounds to the skin preferably certain aminoguanidines dissolved in the polar phase, in combination with a second topically active drug.

Apremilast is insoluble in water. Even in polar solvent phase, only 0.066% of apremilast was found soluble. Therefore, it is challenging to formulate a topical composition of apremilast having an amount, which provides effective concentration of apremilast at the site of action. Hence, there remains an unmet need to develop suitable formulation with favorable characteristics in order to provide desired efficacy with reduced dosage and frequency of administration.

The inventors of the present invention found that topical formulations of Apremilast achieves desired efficacy with site specific drug delivery and thus overcomes GI related side effects. Any specific excipients such as alpha-hydroxy acids (including lactic acid) are not required in the composition. Topical formulations include cream, ointment, gel, transdermal formulations, foam, spray, lotion, solution, emulsion, or suspension. Topical pharmaceutical compositions can be used for the treatment of skin diseases such as Psoriatic arthritis, Plaque psoriasis and atopic dermatitis and inflammatory skin disease condition. Also, it can be efficacious for the treatment of dermatomycosis, scleroderma, epidermolysis bullosa, eczema and systemic lupus erythematous affecting skin.

### SUMMARY OF THE INVENTION

The present invention provides topical pharmaceutical compositions comprising Apremilast and one or more pharmaceutical excipient(s).

First aspect of the present invention provides topical pharmaceutical composition comprising Apremilast in an amount of about 0.1 to 5% w/w of the total composition and one or more pharmaceutical excipient(s).

Another aspect of the present invention provides topical pharmaceutical composition comprising Apremilast in an amount of about 0.1 to 5% w/w of the total composition and a pharmaceutical excipient selected from solublizer, penetration enhancer or mixture thereof.

Another aspect of present invention provides topical pharmaceutical composition comprising Apremilast in an amount of about 0.1 to 5% w/w of the total composition, one or more solublizer(s) and one or more penetration enhancer(s), wherein the solublizer and penetration enhancer are present in a ratio of about 1:0.1 to 1:5.

Another aspect of the present invention is to provide a process of preparation of topical pharmaceutical compositions comprising Apremilast and one or more pharmaceutical excipient(s).

Another aspect of the present invention is to provide a method for treating skin diseases selected from Psoriatic arthritis, Plaque psoriasis, atopic dermatitis, dermatomycosis, scleroderma, epidermolysis bullosa, eczema and systemic lupus erythematous affecting skin by administering topical pharmaceutical composition comprising Apremilast and one or more pharmaceutical excipient(s).

### BRIEF DESCRIPTION OF THE DRAWINGS

FIG. 1. Effect of treatment with Topical formulation of apremilast (Example 3c and 3a) on ear thickness post 24 hours of challenge with FITC.

FIG. 2. Histology of ear of mice administered a) placebo b) formulation of example 3a

FIG. 3. Pharmacokinetics of topical composition of Apremilast in dermis of Female Balb C Mice (Example 3a)

### DETAILED DESCRIPTION OF THE INVENTION

The following paragraphs detail various embodiments of the invention. For the avoidance of doubt, it is specifically intended that any particular feature(s) described individually in any one of these paragraphs (or part thereof) may be combined with one or more other features described in one or more of the remaining paragraphs (or part thereof). In other words, it is explicitly intended that the features described below individually in each paragraph (or part thereof) represent important aspects of the invention that may be taken in isolation and combined with other important aspects of the invention described elsewhere within this specification as a whole, and including the examples and figures. The skilled person will appreciate that the invention extends to such combinations of features and that these have not been recited in detail here in the interests of brevity.

The use of the terms “a” and “an” and “the” and similar referents in the context of describing the invention (especially in the context of the following claims) are to be construed to cover both the singular and the plural, unless otherwise indicated herein or clearly contradicted by context.

The term “Apremilast” as used herein means Apremilast and its pharmaceutically acceptable salts in various solid state forms such as polymorphs, solvates and hydrates. Also, it refers to various isomers and enantiomers of Apremilast, specifically S-form and its pharmaceutically acceptable salts. Apremilast remains in substantially solubilized form in the composition. Preferably composition comprises at least 50% solubilized form, more preferably composition comprises at least 70% solubilized form, most preferably composition comprises at least 90% solubilized form of apremilast. Un-solubilized fraction or crystals can be of present in any known polymorphic form, hydrates or solvates for example, polymorphic forms as disclosed in WO2009120167. Particle size of apremilast may vary from 1 microns to 100 microns (D90), as measured using Laser Diffraction method.

The term “w/w” as used herein means weight of component by total weight of composition, unless specified otherwise. For foamable composition, w/w excludes weight of propellant in total composition.

The term “about” as used herein means plus or minus 30%, preferably 20%, most preferably 10%, of the numerical value of the number with which it is being used.

The term “aqueous composition” as used herein means water is added into the composition during its preparation. Any amount of water can be added as per requirement of the type of the topical composition. Preferably, from at least 10% to upto 90% w/w of water is added in the composition.

Inventors of the present invention have surprisingly found that topical composition of Apremilast in comparison with oral formulation can lead to dose reduction, provides improved efficacy because of site specific availability of desired concentration of drug as well as minimize the adverse reactions associated with oral administration. Besides these, it also provides improved patient compliance and enhanced psychological impact.

The etiology of Psoriasis disease condition involves different inflammatory mediators, which are triggered by external stimuli from the skin and the genetic factors. Therefore, in Psoriasis site specific delivery as well as systemic delivery is required. Preferred topical composition will cater the delivery needs for the inflammatory condition. However, for desired efficacy, some minimum concentration of active agent in dermis or penetration through skin is required. Present invention provides a topical composition comprising Apremilast in an amount of about 0.1 to 5% w/w of the total composition. It was observed that, this specified amount is required for desired concentration of Apremilast in the dermis, for drug to be effective. Composition of present invention provides improved concentration in dermis as compared to oral composition.

Since, apremilast is insoluble in water, it is difficult to formulate it in a suitable formulation, specifically when used in higher concentration for topical formulation. The drug remains undissolved in the solution or gets precipitated and crystals are observed. Hence, to overcome this problem, the inventors developed topical composition comprising one or more suitable pharmaceutical excipients which includes solubilizers, surfactants, co solvents, crystal growth inhibitors, Penetration enhancer etc. It was surprisingly found that specific combination of selected solubilizers and penetration enhancers/crystal growth inhibitors in a selected range resulted into substantially complete dissolution of Apremilast and provides a clear stable topical composition comprising Apremilast in an amount of about 0.1 to 5% by weight of total composition.

First embodiment of the present invention provides topical pharmaceutical compositions comprising Apremilast in an amount of 0.1 to 5% w/w of the total composition and one or more pharmaceutical excipient(s).

Another embodiment of the present invention provides topical pharmaceutical composition comprising Apremilast, and a pharmaceutical excipient selected from solublizer, penetration enhancer or mixture thereof.

Another embodiment of compositions comprising Apremilast in an amount of about 0.1 to 5% w/w of the total composition and solublizer or penetration enhancer. Composition comprises solublizer is in amount of 5-30% w/w of total composition. Most preferably solublizer is in amount of 5-25% of total weight of composition. Composition comprises penetration enhancer is in amount of 1-50% w/w of total composition. Most preferably solublizer is in amount of 1-40% of total weight of composition.

Another embodiment of the present invention provides topical pharmaceutical composition comprising Apremilast, and a solublizer wherein apremilast and solublizer are present in ratio of about 1:1 to 1:50. Preferably, apremilast and solublizer are present in ratio of about 1:5 to 1:50.

Solublizers according to the present invention includes but not limited to dimethyl malonate, diethyl succinate, diethyl glutarate, diethyl adipate, dipropyl adipate, dibutyl sebacate, diisopropyl sebacate, diethyl pimelate, diethyl suberate, diethyl azelate, dibutyl adipate, dibutyl sebacate, methyl ethyl succinate, diethyl ethyl-isopropylmalonate, diethyl isosuccinate, benzyl alcohol, benzyl benzoate, cyclodextrin, glycerine monostearate, lecithin, butylene glycol, dibutyl phthalate, diethyl phthalate, dimethyl ether, diethyl ether, ethyl acetate, ethyl lactate, ethyl oleate, glycofurol, isopropyl alcohol, triacetin, triethanolamine, hexylene glycol, dimethyl sulfoxide (DMSO) and/or dimethyl isosorbide. Preferably, dimethyl isosorbide is used. Solublizer according to the present invention is present in an amount ranging from 5-30% w/w of the composition. Preferably, it is present in an amount ranging from 5-25% w/w of the composition.

Penetration enhancers act by various mechanisms to reduce the skin barrier and accelerate drug absorption through skin. Penetration enhancers according to the present invention includes but not limited to sulphoxides such as dimethylsulphoxide (DMSO), Dodecyl methyl sulfoxide; Azones such as 1-Butyl-2-azacycloheptanones, 1-Hexyl-2-azacycloheptanones, 1-Octyl-2-azacycloheptanones, 1-dodecylazacycloheptan-2-one (Laurocapram); pyrrolidones such as 1-Ethyl-2-pyrrolidone, 1-Butyl-2-pyrrolidones, 1-Hexyl-2-pyrrolidinone, 1-Octyl-2-pyrrolidinone, 1-Decyl-2-pyrrolidinone, 1-Dodecyl-2-pyrrolidinone, N-methyl-2-pyrrolidone, 2-pyrrolidone; alcohols such as 1-butanol, 1-pentanol, 1-hexanol, 1-octanol, benzyl alcohol, 2-phenylethanol, ethanol, isopropyl alcohol, decanol, Caprylic alcohol, hexylene glycol, butylene glycol; Dioxolane derivatives such as 2-(1-Butyl)-2-methyl-1,3 dioxolane, 2-(1-Hexyl)-2-methyl-1,3 dioxolane, 2 N-nonyl-1,3-dioxolanes; fatty acids such as Decanoic acid, Undecanoic acid, Lauric acid, Tridecanoic acid, Myristic acid, Pentadecanoic acid, Palmitic acid, Stearic acid, Linoleic acid, Oleic acid, Ricinoleic acid, lauric acid, myristic acid and capric acid; surfactants such as Sodium lauryl sulfate, Cetyltrimethyl amonium bromide, Sorbitan monolaurate, Polysorbate 80, Dodecyl dimethyl ammoniopropane sulfate, polyoxyethylene-2-oleyl ether, polyoxy ethylene-2-stearly ether; terpenes such as Thymol, Menthol, Menthone, Carvacrol, Cineole, limonene; urea, sorbitol, mannitol, oligosaccharides, benzyl benzoate, dimethyl isosorbide, monooleate of ethoxylated glycerides (with 8 to 10 ethylene oxide units), polyethylene glycol 200-600, Alpha-Tocopherol Polyethylene glycol succinate (Vitamin E-TPGS), transcutol (diethylene glycol monoethyl ether), glycofurol (tetrahydrofurfuryl alcohol PEG ether), Caprylocaproyl macrogol-8 glycerides (Labrasol), or mixtures thereof. Preferably, diethylene glycol monoethyl ether (Transcutol)) is used as penetration enhancers. The penetration enhancer(s) may be present in an amount ranging from 1-50% w/w of the composition. Preferably, the penetration enhancer(s) are present in an amount ranging from 1-40% w/w of the composition.

In a preferred embodiment, the present invention provides topical pharmaceutical composition comprising Apremilast, dimethyl isosorbide as solublizer and diethylene glycol monoethyl ether (Transcutol) as penetration enhancer.

A preferred embodiment of the present invention provides topical pharmaceutical composition comprising Apremilast, one or more solublizer(s) and one or more penetration enhancer(s), wherein the solublizer and penetration enhancer are present in a ratio of about 1:0.1 to 1:5.

In another embodiment, the topical pharmaceutical composition of the present invention comprises apremilast as sole active ingredient.

In an alternative embodiment, present invention provides an aqueous topical pharmaceutical composition comprising Apremilast and one or more pharmaceutical excipient(s). Aqueous composition comprises apremilast in an amount of about 0.1 to 5% w/w of the total composition. Pharmaceutical excipients are selected from solublizer, penetration enhancer or mixture thereof.

Aqueous composition of present invention is advantageous as it is easy to manufacture and is water washable after its application, and thus increases ease of application without stickiness.

The topical pharmaceutical compositions according to the present invention further comprises one or more pharmaceutical excipient(s) which includes but not limited to oleaginous bases, absorption bases, emulsifying agents (emulsifiers), preservatives, antioxidants, surfactants, emollients, humectants, gelling agents, thickening agents, stiffening agents, viscosity enhancers, film formers, foam formers, stabilizers, buffering agents, pH adjusting agents, suspending agents, solvents, co-solvents, crystal growth inhibitors, diluents, chelating agents, vehicles, propellants, coloring agents and/or perfumes.

pH of the composition according to present invention ranges from 3-7, preferably it is 4-6. Suitable pH adjusting agent can be added in the composition to maintain the required pH.

Topical pharmaceutical compositions according to the present invention can be in the form of cream, ointment, gel, transdermal formulations, foam, spray, lotion, solution, emulsion or suspension.

A general embodiment of present invention provides process of preparation of topical pharmaceutical compositions comprising apremilast comprising:
a) preparing solution of Apremilast, preferably 0.1-5% of apremilast in one or more pharmaceutical excipients, preferably solubilizers.
b) Optionally mixing solution of step a) with one or more pharmaceutically acceptable excipient(s).
c) Preparing topical pharmaceutical composition in the form of cream, ointment, gel, transdermal formulations, foam, spray, lotion, solution, emulsion or suspension.

### Topical Foamable Composition

In an embodiment, the present invention provides topical foamable composition comprising Apremilast and one or more pharmaceutical excipients which includes but not limited to oil bases, water, co-solvents, solvents, alcohol, solubilizers, emulsifiers, penetration enhancers, foam formers, surfactants, foam stabilizers, preservatives, film forming agents, crystal growth inhibitors, gelling agents, emollients and/or propellants. Preferred excipients for topical foamable composition are one or more solublizer, penetration enhancer and crystal growth inhibitor. Additionally, topical foamable composition comprises solvent, co-solvent, surfactant, foam stabilizer and preservatives.

In an another preferred embodiment, the topical foamable composition according to the present invention can form an aqueous foam, hydro alcoholic foam, emulsion foam, micro emulsion foam, ointment foam, oil foam or saccharide foam.

In a more preferred embodiment, the present invention provides micro emulsion foamable composition comprising Apremilast and one or more pharmaceutical excipient(s) which includes but not limited to oil bases, water, co-solvents, solubilizers, emulsifiers, penetration enhancers, foam formers, surfactants, foam stabilizers, preservatives, film forming agents, crystal growth inhibitors, emollients and/or propellants.

In the most preferred embodiment, the present invention provides micro emulsion foamable composition comprising Apremilast and glyceryl monocaprylate as oil base as well as emollient, purified water as vehicle, PEG-400 as co-solvent, dimethyl isosorbide as solublizer as well as emulsifier, diethylene glycol monoethyl ether as penetration enhancer, ceteareth as surfactant as well as foam stabilizer, cetyl alcohol as foam stabilizer, polyethylene glycol (15)-hydroxy stearate as surfactant as well as solublizer, phenoxy ethanol as preservative, polyvinyl pyrrolidone as film forming agent as well as crystal growth inhibitor and/or deodorized liquefied n-butane and/or propane gas as propellant.

Preferably, amount of Apremilast in topical foamable composition is from 0.1-5% and solublizer and penetration enhancer are present in a ratio of about 1:0.1 to 1:5. More preferably, the ratio of solublizer to penetration enhancer is about 1:0.5.

A topical foamable composition can be prepared by any known process, preferably in the form of a solution comprising one or more propellant and filled in suitable container for foam delivery.

Another embodiment of present invention provides a pharmaceutical product comprising a) topical foamable composition comprising apremilast and one or more pharmaceutical excipients(s) b) a suitable container for foam delivery.

Another embodiment of the present invention provides process for preparation of topical foamable composition, preferably micro emulsion, composition comprising:
a) Preparing oil phase by melting and mixing one or more pharmaceutical excipient(s),
b) adding a solution of Apremilast in one or more solubilizers and/or penetration enhancer to the oil phase prepared in step a),
c) preparing an aqueous phase by dissolving one or more suitable pharmaceutical excipients(s),
d) preparing a micro emulsion by adding the aqueous phase prepared in step c) to the oil phase prepared in step b),
e) adding a suitable propellant to the micro emulsion prepared in step d).

In a preferred embodiment, the present invention provides process for preparation of micro emulsion foamable composition comprising:
a) Preparing oil phase by melting and mixing one or more pharmaceutical excipient(s) comprising surfactants, co-solvents, preservatives, oil base, foam stabilizers and/or emollients,
b) adding a solution of Apremilast in one or more solubilizers and/or penetration enhancer to the oil phase prepared in step a),
c) preparing an aqueous phase by dissolving one or more crystal growth inhibitors,
d) preparing a micro emulsion by adding the aqueous phase prepared in step c) to the oil phase prepared in step b),
e) adding a suitable propellant which includes liquefied and/or compressed gas to the micro emulsion prepared in step d).

A more preferred embodiment of the present invention provides process for preparation of micro emulsion foam composition comprising:
a) Preparing oil phase by melting and mixing polyethylene glycol (15)-hydroxy stearate, ceteareth, cetyl alcohol, polyethylene glycol, glyceryl monocaprylate and phenoxy ethanol,
b) adding a solution of Apremilast in dimethyl isosorbide and diethylene glycol monoethyl ether to the oil phase prepared in step a),
c) preparing an aqueous phase by dissolving polyvinyl pyrrolidone in water,
d) preparing a micro emulsion by adding the aqueous phase prepared in step c) to the oil phase prepared in step b),
e) adding deodorized liquefied n-butane and/or propane gas to the micro emulsion prepared in step d) and filling in aluminium cans.

### Topical Ointment Composition

In an another preferred embodiment, the topical pharmaceutical composition according to the present invention provides topical ointment comprising Apremilast and one or more pharmaceutical excipients which includes but not limited to ointment bases such as oleaginous bases, absorption bases, water removable bases, water soluble bases; preservatives, anti oxidants, chelating agents, emulsifying agents, solubilizers, penetration enhancers and/or perfumes. Preferred excipients for topical ointment composition are one or more solublizer, penetration enhancer and ointment base. Additionally, ointment composition may comprise surfactant and viscosity enhancer.

In a more preferred embodiment, the topical ointment comprises Apremilast and dimethyl isosorbide as solublizer, diethylene glycol monoethyl ether (Transcutol) as penetration enhancer, polyethylene glycol (PEG) 540 blend as ointment base, polysorbate 60 as surfactant as well a solublizer and polyethylene glycol as viscosity enhancer. More preferably, Polyethylene glycol 6000 is used as viscosity enhancer.

Preferably, amount of Apremilast in topical ointment composition is from 0.1-5% and solublizer and penetration enhancer are present in a ratio of about 1:0.1 to 1:5. More preferably, the ratio of solublizer to penetration enhancer is about 1:0.25 to 1:1.

In an another preferred embodiment, the present invention provides process for preparation of ointment composition comprising:
a) preparing a solution by melting and mixing one or more ointment base(s) and one or more pharmaceutically excipients,
b) preparing drug solution by dissolving Apremilast in one or more solubilizers(s),
c) adding drug solution prepared in step b) to the solution prepared in step a).

In a preferred embodiment, the present invention provides process for preparation of ointment composition comprising:
a) preparing a solution by melting and mixing one or more ointment base(s) and one or more pharmaceutical excipients which includes penetration enhancer, surfactant and/or viscosity enhancer,
b) preparing drug solution by dissolving Apremilast in one or more solubilizers(s),
c) adding drug solution prepared in step b) to the solution prepared in step a).

In a more preferred embodiment, the present invention provides process for preparation of ointment composition comprising:
a) preparing a solution by melting and mixing polyethylene glycol 540, Polysorbate 60, polyethylene glycol 6000 and diethylene glycol monoethyl ether,
b) preparing drug solution by dissolving Apremilast in dimethyl isosorbide,
c) adding drug solution prepared in step b) to the solution prepared in step a).

### Topical Cream Composition

In an another preferred embodiment, the topical pharmaceutical composition according to the present invention provides topical cream comprising Apremilast and one or more pharmaceutical excipients which includes but not limited to oleaginous bases, absorption bases, water removable bases, water soluble bases; surfactants preservatives, anti oxidants, chelating agents, emulsifiers, solubilizers, stiffening agents, penetration enhancers, vehicles and/or perfumes. Preferred excipients for topical cream composition are one or more solublizer and penetration enhancer. Additionally, cream composition may comprise oil base, emulsifier, stiffening agent, surfactant and preservative.

In a more preferred embodiment, the topical cream comprises Apremilast, light mineral oil as oil base, glyceryl stearate as emulsifier, sorbitan monostearate, cetyl alcohol and Ceteareth (particularly Ceteareth 20) as emulsifier and stiffening agent, stearyl alcohol and stearic acid as stiffening agent, diethylene glycol monoethyl ether (Transcutol) as penetration enhancer, dimethyl isosorbide as solublizer, polysorbate as surfactant, methyl paraben and propyl paraben as preservative and purified water as vehicle.

Preferably, amount of Apremilast in topical cream composition is from 0.1-5% and solublizer and penetration enhancer are present in a ratio of about 1:0.1 to 1:5. More preferably, the ratio of solublizer to penetration enhancer is about 1:0.5 to 1:1.

In an another preferred embodiment, the present invention provides process for preparation of cream composition comprising:
a) preparing oil phase by melting and mixing one or more oil base(s) and one or more pharmaceutical excipients,
b) preparing drug solution by dissolving Apremilast in one or more solubilizers(s),
c) preparing water phase by dissolving one or more surfactant,
d) adding water phase prepared in step c) into oil phase prepared in step a) to form emulsion,
e) adding drug solution prepared in step b) to the emulsion prepared in step d).

In a preferred embodiment, the present invention provides process for preparation of cream composition comprising:
a) preparing oil phase by melting and mixing one or more oil base(s) and one or more pharmaceutical excipients which includes emulsifier, stiffening agents, preservative and/or penetration enhancer,
b) preparing drug solution by dissolving Apremilast in one or more solubilizers(s),
c) Preparing water phase by dissolving one or more surfactant,
d) adding water phase prepared in step c) into oil phase prepared in step a) to form emulsion
e) adding drug solution prepared in step b) to the emulsion prepared in step d).

In a more preferred embodiment, the present invention provides process for preparation of cream composition comprising:
a) preparing oil phase by melting and mixing light mineral oil, glyceryl stearate, sorbitan monostearate, cetyl alcohol, stearyl alcohol, stearic acid, ceteareth 20, diethylene glycol monoethyl ether, methyl paraben and propyl paraben,
b) preparing drug solution by dissolving Apremilast in dimethyl isosorbide,
c) Preparing water phase by dissolving Polysorbate 60,
d) adding water phase prepared in step c) into oil phase prepared in step a) to form emulsion
e) adding drug solution prepared in step b) to the emulsion prepared in step d).

### Topical Gel Composition

In an another preferred embodiment, the topical pharmaceutical composition according to the present invention provides topical gel comprising Apremilast and one or more pharmaceutical excipients which includes but not limited to gelling agents, solubilizers, preservatives, pH adjusting agents, buffering agents, and/or vehicles. Preferred excipients for topical gel composition are one or more solublizer, gelling agent and penetration enhancer. Additionally, gel composition may comprise preservative and vehicle.

In a more preferred embodiment, the topical gel comprises Apremilast, polyacrylic acid polymers, as gelling agent, diethylene glycol monoethyl ether as penetration enhancer, dimethyl isosorbide as solublizer, methyl paraben as preservative, sodium hydroxide as pH adjusting agent and purified water as vehicle.

Preferably, amount of Apremilast in topical cream composition is from 0.1-5% and solublizer and penetration enhancer are present in a ratio of about 1:0.1 to 1:5. More preferably, the ratio of solublizer to penetration enhancer is about 1:5.

In an another embodiment, the present invention provides process for preparation of gel composition comprising:
a) preparing an aqueous solution by dissolving one or more pharmaceutical excipient(s) in water,
b) preparing drug solution by dissolving Apremilast in one or more solubilizers(s) and/or one more more penetration enhancer(s),
c) adding drug solution prepared in step b) to the solution prepared in step a).

In a preferred embodiment, the present invention provides process for preparation of gel composition comprising:
a) preparing an aqueous solution by dissolving gelling agent and preservative in water,
b) preparing drug solution by dissolving Apremilast in one or more solubilizers(s),
c) adding drug solution prepared in step b) to the solution prepared in step a) and adjusting the pH using pH adjusting agent.

In a more preferred embodiment, the present invention provides process for preparation of gel composition comprising:
a) preparing an aqueous solution by dissolving polyacrylic acid polymers (carbomer homopolymer type C), and methyl paraben in water,
b) preparing drug solution by dissolving Apremilast in dimethyl isosorbide and diethylene glycol monoethyl ether,
c) adding drug solution prepared in step b) to the solution prepared in step a) and adjusting the pH using sodium hydroxide.

### Excipients

Co-solvents according to the present invention includes but not limited to azone (1-dodecylazacycloheptan-2-one), 2-(n-nonyl)-1,3-dioxolane; polyols such as, hexylene glycol, diethylene glycol, propylene glycol, n-alkanols, terpenes, di-terpenes, tri-terpenes, terpen-ols, limonene, 1-menthol, dioxolane, ethylene glycol, other glycols; sulfoxides, such as dimethylsulfoxide (DMSO), dimethylformanide, methyl dodecyl sulfoxide, dimethylacetamide, monooleate of ethoxylated glycerides (with 8 to 10 ethylene oxide units), esters such as ethyl acetate, butyl acetate, methyl proprionate, capric/caprylic triglycerides, octyl myristate, dodecyl-myristate; myristyl alcohol, lauryl alcohol, lauric acid, lauryl lactate ketones; amides such as acetamide oleates such as triolein; various alkanoic acids such as caprylic acid; lactam compounds such as azone; alkanols such as dialkylamino acetates. More preferably, polyethylene glycol derivatives which are liquid at room temperature including PEG-200, PEG-300, PEG-400 and/or PEG-600 are used as co-solvents. Most preferably, PEG-400 is used as co-solvent. The co-solvent(s) may be present in an amount ranging from 5-70% w/w of the composition. Preferably, it is present in an amount ranging from 20-50% w/w of the composition.

Oil bases according to the present invention aids in softening the skin and imparts soothing effect. Hence, they also play an important role as emollient in topical formulation such as micro emulsion foam formulation or creams. Oil bases according to the present invention includes but not limited to mineral oils such as liquid paraffin and liquid petrolatum; oils of plant origin such as jojoba oil, sesame oil, rapeseed oil; synthetic oils such as fatty esters, for instance purcellin oil, 2-ethylhexyl palmitate, 2-octyldodecyl stearate, 2-octyldodecyl erucate, isostearyl isostearate, 2-octyldodecyl benzoate and hydrogenated isoparaffin; triglycerides of caprylic/capric acids, octyldodecanol, and isohexadecane. Preferably, light mineral oil or glyceryl monocaprylate (Capmul MCM) is used. The oil base may be present in an amount ranging from 1-30% w/w of the composition. Preferably, it is present in an amount ranging from 5-25% w/w of the composition.

Capmul MCM (glyceryl monocaprylate) used as oil base and emollient herein, also plays an important role as penetration enhancer and surfactant.

Preservatives according to the present invention includes but not limited to benzalkonium chloride, methyl, ethyl, propyl or butyl paraben, benzyl alcohol, phenylethyl alcohol, benzethonium, chlorobutanol, Phenoxy ethanol, potassium sorbate, benzoic acid or mixtures thereof. Preferably, phenoxy ethanol is used. The preservative(s) may be present in an amount ranging from 0.1-10% w/w of the composition. Preferably, it is present in an amount ranging from 0.5-5% w/w of the composition.

Surfactants according to the present invention includes but not limited to anionic, cationic, non-ionic, zwitterionic and/or amphoteric surfactants. Non limiting examples include anionic sufactants such as sodium lauryl sulfate, ammonium lauryl sulfate, sodium lauryl ether sulfate, triethylamine lauryl sulfate, triethanolamine lauryl sulfate, monoethanolamine lauryl sulfate, docusate sodium, and/or potassium lauryl sulfate; cationic surfactants such as cetrimonium bromide, Benzalkonium chloride and/or stearyl dimethylbenzyl ammonium chloride; non-ionic surfactants such as fatty alcohols, cetyl alcohol, stearyl alcohol, cetostearyl alcohol, oleyl alcohol, Polyoxyethylene glycol sorbitan alkyl esters (Polysorbate, Tween), Sorbitan alkyl esters (Spans), sorbitan monostearate, stearic acid, Block copolymers of polyethylene glycol and polypropylene glycol (Poloxamers), polyoxyethylene lauryl amine, PEG-distearate, PEG-hydroxystearate, propylene glycol monostearate; Polyoxyethylene_glycol alkyl ethers (Brij) such as polyoxyethylene butyl ether, polyoxyethylene cetyl ether, Polyethyleneglycol cetyl/stearyl ether (Ceteareth), polyoxyethylene oleyl cetyl ether, polyoxyethylene oleyl ether, laureth-4 and/or polyoxyethylene lauryl ether.

Surfactants also enhance solubilization of drug compound and/or other excipients in the formulation and play a critical role as solubilizers. Further, surfactants also function as foam stabilizing agent by increasing longevity of foam and improving foam appearance. Preferably, the present invention comprises of Polyethyleneglycol cetyl/stearyl ether (Ceteareth) as surfactant as well as foam stabilizer, cetyl alcohol as foam stabilizer and PEG-hydroxystearate (Solutol) as a surfactant as well as solublizer.

In addition to these, certain surfactants also play an important role as emulsifiers in a topical composition in order to stabilize the emulsion system. Surfactants particularly, ceteareth, cetyl alcohol, stearyl alcohol and sorbitan monostearate behave as emulsifiers.

In topical compositions such as cream or ointment, surfactants act as stiffening agent for the formulation. Surfactants, particularly sorbitan monostearate, Ceteareth, stearic acid and fatty alcohols such as cetyl alcohol and stearyl alcohol act as stiffening agent for topical cream formulation.

Surfactants according to the present invention may be present in an amount ranging from 0.5-25% w/w of the composition. Preferably, it is present in an amount ranging from 5-20% w/w of the composition.

Film forming agents according to the present invention include but not limited to hydroxypropyl methyl cellulose (Hypromellose), hydroxypropyl cellulose, polyvinyl pyrrolidone (PVP), gelatin, polyethylene oxide, hydroxyethyl cellulose, sodium alginate and mixture thereof. Preferably, PVP-K30 is used. Film forming agent is present in an amount ranging from 2-15% w/w of the composition. Preferably, it is present in an amount ranging from 2-10% w/w of the composition.

Crystal growth inhibitors according to the present invention includes but not limited to polymers such as hydroxypropyl methylcellulose (HPMC), hydroxypropyl methylcellulose acetate succinate (HPMCAS), carboxymethyl cellulose acetate butyrate (CMCAB), poly (vinyl pyrollidinone) (PVP), poly (vinyl pyrollidinone-vinyl acetate) (PVP/VA), cellulose acetate adipate propionate (CAAdP). CAPH (cellulose acetate phthalate), HPMCPH (hydroxypropylmethylcellulose phthalate), CASub (cellulose acetate suberate), CASeb (cellulose acetate sebacate), and/or PEG (polyethylene glycol). Preferably, PVP-K30 is used. Crystal growth inhibitor is present in an amount ranging from 2-15% w/w of the composition. Preferably, it is present in an amount ranging from 2-10% w/w of the composition.

PVP-K30 according to the present invention plays a dual role as film-forming agent as well as crystal growth inhibitor and prevents precipitation of Apremilast and/or excipients in the composition.

pH adjusting agent according to the present invention includes but not limited to an acid, an acid salt, basic inorganic salts, organic bases such as an alkylamine (trimethylamine, triethylamine and the like), a heterocyclic amine (pyridine, picoline and the like), an alkanolamine (ethanolamine, diethanolamine, triethanolamine and the like), meglumine, dicyclohexylamine, N,N′-dibenzylethylenediamine and a basic amino acid (arginine, lysine, ornithine and the like); bicarbonates, carbonates, and hydroxides such as alkali or alkaline earth metal hydroxide as well as transition metal hydroxides such as sodium hydroxide and potassium hydroxide. Further, the pH adjusting agent can also be a buffer. Suitable buffers include citrate/citric acid buffers, acetate/acetic acid buffers, phosphate/phosphoric acid buffers, formate/formic acid buffers, propionate/propionic acid buffers, carbonate/carbonic acid buffers, ammonium/ammonia buffers, and the like. Preferably, sodium hydroxide is used as a pH adjusting agent.

Gelling agents according to present invention include sugars or sugar derived alcohols, starch and starch derivatives, cellulose derivatives, gums, carbomers and polyacrylic acid polymers (Carbopol®, preferably Carbopol® 980), polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof.

Ointment bases according to the present invention includes oleaginous bases such as petrolatum, white/yellow petrolatum, white ointment; absorption bases such as lanolin, anhydrous lanolin, cold cream; water removable bases such as hydrophilic ointments, vanishing creams and water soluble bases such as polyethylene glycol (PEG). Preferably, PEG is used as ointment base.

Viscosity enhancer according to present invention include naturally-occurring polymeric materials such as, locust bean gum, sodium alginate, sodium caseinate, egg albumin, gelatin agar, carrageenan gum sodium alginate, xanthan gum, quince seed extract, tragacanth gum, starch, chemically modified starches and the like; semi-synthetic polymeric materials such as cellulose ethers (e.g. hydroxyethyl cellulose, methyl cellulose, carboxymethyl cellulose, hydroxy propyl methyl cellulose), polyvinylpyrrolidone, polyvinyl alcohol, guar gum, hydroxypropyl guar gum, soluble starch, cationic celluloses, cationic guars and the like; synthetic polymeric materials such as carboxyvinyl polymers, polyvinylpyrrolidone, polyvinyl alcohol polyacrylic acid polymers, polymethacrylic acid polymers, polyvinyl acetate polymers, polyvinyl chloride polymers, polyvinylidene chloride polymers; polyethylene glycols and the like. Preferably, polyethylene glycol is used as viscosity enhancer.

Propellants according to the present invention includes but not limited to chlorofluorocarbons such as Trichloromonofluoromethane, Dichlorodifluoromethane, Dichlorotetrafluoroethane; hydrocarbons such as propane, butane, isopropane; hydrochlorofluorocarbons or hydrofluorocarbons such as Chlorodifluoromethane, Trifluoromonofluoroethane, Difluoroethane, Chlorodifluoroethane, Heptafluoropropane; compressed gases such as nitrous oxide, nitrogen carbon dioxide or admixtures thereof. More preferably, propane and/or butane in liquefied form are used as propellants. Most preferably, deodorized liquefied petroleum gas is used. The propellant(s) may be present in an amount ranging from 3-25 w/w % of the composition. Preferably, the propellant(s) are present in an amount ranging from 5-15% w/w of the composition.

Compositions according to present invention may optionally further comprise one or more stabilizer, anti oxidants, coloring agents, fragrances and the like. Example and suitable amount of said optional excipient is known to a skilled person or as given in Handbook of pharmaceutical excipients (sixth edition, 2009).

Another embodiment of the present invention is to provide method for treating skin diseases selected from Psoriatic arthritis, Plaque psoriasis, atopic dermatitis, dermatomycosis, scleroderma, epidermolysis bullosa, eczema and systemic lupus erythematous using topical pharmaceutical compositions comprising Apremilast and one or more pharmaceutical excipient(s).

Another preferred embodiment of the present invention is to provide a method of treating skin diseases selected from Psoriatic arthritis, Plaque psoriasis, atopic dermatitis, dermatomycosis, scleroderma, epidermolysis bullosa, eczema and systemic lupus erythematous using micro emulsion foam composition comprising Apremilast and one or more pharmaceutical excipient(s).

The invention will be further illustrated by the following examples, however, without restricting the scope to these embodiments.

### Example 1 (Apremilast Topical Foamable Composition)

TABLE 1

| Sr. | | 1a | 1b |
|---|---|---|---|
| No. | Ingredients | % w/w | % w/w |
| 1. | Apremilast | 2 | 2 |
| 2. | Diethylene glycol monoethyl ether (Transcutol) | — | 7.5 |
| 3. | Dimethyl isosorbide | 10 | 15 |
| 4. | Ceteareth 20 | 5 | 5 |
| 5. | PEG-400 | — | 30 |
| 6. | Polysorbate 80 | 2.5 | — |
| 7. | Polyethylene glycol (15) - | — | 10 |
| | hydroxy stearate (Solutol HS 15) | | |
| 8. | Phenoxy ethanol | — | 0.5 |
| 9. | Glyceryl monocaprylate (Capmul MCM) | — | 10 |
| 10. | Povidone (PVP-K30) | 2 | — |
| 11. | Glycerine | 10 | — |
| 12. | Hexylene glycol | 25 | — |
| 13. | Purified water | 43.5 | 20 |
| Total | | 100.00 | |
| 14. | Deodorized Liquefied Petroleum Gas (LPG) | — | 8 |

### Process for the Preparation of Apremilast Topical Foamable Formulation

Apremilast was solubilized in dimethyl isosorbide and/or Diethylene glycol monoethyl ether (Transcutol) to obtain drug solution. Polyethylene glycol (15)-hydroxy stearate, Ceteareth 20, Cetyl alcohol, PEG-400, Glyceryl monocaprylate, polysorbate and Phenoxy ethanol as per table 1, were melted and mixed to prepare oil phase. The drug solution previously prepared was added dropwise to the oil phase and stirred to obtain mixture. PVP-K30 in Example 1a was added in water to prepare an aqueous phase and water was used as aqueous phase in Example 1b. The aqueous phase was added to the prepared mixture and stirred slowly to obtain a micro emulsion. Micro emulsion so prepared was filled in aluminum cans and propellant, Deodorized Liquefied Petroleum Gas (LPG) was added in Example 1b to obtain topical foamable composition.

### Example 2 (Apremilast Topical Foamable Composition)

TABLE 2

| Sr. | | 2a | 2b |
|---|---|---|---|
| No. | Ingredients | % w/w | % w/w |
| 1. | Apremilast | 2 | 2 |
| 2. | Diethylene glycol monoethyl ether (Transcutol) | — | 8 |
| 3. | Dimethyl isosorbide | 15 | 15 |
| 4. | Ceteareth 20 | 5 | 5 |
| 5. | Cetyl alcohol | 0.5 | 0.5 |
| 6. | PEG-400 | 30 | 30 |
| 7. | Polyethylene glycol (15) - | 10 | 10 |
| | hydroxy stearate (Solutol HS 15) | | |
| 8. | Phenoxy ethanol | 0.5 | 0.5 |
| 9. | Glyceryl monocaprylate (Capmul MCM) | 10 | 10 |
| 10. | Povidone (PVP-K30) | 5 | 5 |
| 11. | Purified water | 22 | 14 |
| Total | | 100 | 100.00 |
| 12. | Deodorized Liquefied Petroleum Gas (LPG) | — | 8 |

### Process for the Preparation of Apremilast Topical Foamable Formulation

Apremilast was solubilized in mixture of dimethyl isosorbide and/or Diethylene glycol monoethyl ether (Transcutol) to obtain drug solution. Other excipients such as Polyethylene glycol (15)-hydroxy stearate, Ceteareth 20, Cetyl alcohol, PEG-400, Glyceryl monocaprylate, and Phenoxy ethanol were melted and mixed to prepare oil phase. The drug solution previously prepared was slowly added dropwise to the oil phase and stirred to obtain uniform mixture. PVP-K30 was added in water to prepare an aqueous phase. The aqueous phase was added to the prepared uniform mixture and stirred slowly to obtain a micro emulsion. Micro emulsion so prepared was filled in aluminum cans and finally propellant, Deodorized Liquefied Petroleum Gas (LPG) was added to obtain topical micro emulsion foam of Apremilast.

### Example 3 (Apremilast Topical Foamable Composition)

TABLE 3

| Sr | | 3a | 3b | 3c | 3d |
|---|---|---|---|---|---|
| No | Ingredients | % (w/w) | % (w/w) | % (w/w) | % (w/w) |
| 1 | Apremilast | 3 | 1 | 0.3 | 0.3 |
| 2 | Dimethyl | 15 | 15 | 15 | 15 |
| | isosorbide | | | | |
| 3 | Cetaerth-20 | 5 | 5 | 5 | 5 |
| 4 | Cetyl alchol | 0.50 | 0.50 | 0.50 | 1.5 |
| 5 | Stearyl alcohol | — | — | — | 3 |
| 6 | Glyceryl Caprylate/ | 10 | 10 | 10 | 10 |
| | Caprate (Capmul | | | | |
| | MCM) | | | | |
| 7 | Diethylene glycol | 8 | 8 | 8 | 8 |
| | monoethylether | | | | |
| | (Transcutol) | | | | |
| 8 | Polyethylene | 30 | 30 | 30 | 30 |
| | glycol -400 | | | | |
| 9 | Macrogol15- | 10 | 10 | 10 | 10 |
| | hydroxy stearate | | | | |
| | (Solutol HS 15) | | | | |
| 10 | Povidone K30 | 5 | 5 | 5 | 5 |
| 11 | Water | 13 | 15 | 15.7 | 8.2 |
| 12 | Phenoxyethanol | 0.50 | 0.50 | 0.50 | 1.0 |
| | Total | 100 | 100 | 100 | 100 |

Compositions of Example 3a-3d were prepared according to the process described in Example 2.

### Example 4 (Apremilast Ointment)

TABLE 4

| | | Exam- | Exam- | Exam- | Exam- |
|---|---|---|---|---|---|
| Sr | | ple 4a | ple 4b | ple 4c | ple 4d |
| No | Ingredients | % (w/w) | % (w/w) | % (w/w) | % (w/w) |
| 1 | Apremilast | 2 | 2 | 0.9 | 0.3 |
| 2 | Dimethyl | 15 | 15 | 15 | 15 |
| | isosorbide | | | | |
| 3 | Transcutol | 8 | 8 | 8 | 4 |
| 4 | PEG 540 Blend | 73 | 71 | 69.1 | 73.7 |
| 5 | Polysorbate 60 | 2 | 2 | 2 | 2 |
| 6 | PEG 6000 | — | 2 | 5 | 5 |
| | Total | 100 | 100 | 100 | 100 |

### Process for the Preparation of Apremilast Topical Ointment

PEG-540 blend, polysorbate 60, PEG 6000 and Transcutol were melted and mixed to prepare a homogenous solution. Apremilast was dissolved in dimethyl isosorbide to prepare drug solution, which was added dropwise to the solution prepared in previous step to obtain uniform mixture for topical ointment.

### Example 5 (Apremilast Ointment)

TABLE 5

| Sr | | Example 5 |
|---|---|---|
| No | Ingredient | % (w/w) |
| 1 | Apremilast | 0.6 |
| 2 | Dimethyl isosorbide | 15 |
| 3 | Transcutol | 8 |
| 4 | PEG 540 Blend | 69.4 |
| 5 | Polysorbate 60 | 2 |
| 6 | PEG 6000 | 5 |
| | Total | 100 |

Topical Ointment formulation was prepared according to the process described in Example 4.

### Example 6 (Apremilast Ointment)

TABLE 6

| Sr | | Example 6 |
|---|---|---|
| No | Ingredient | % (w/w) |
| 1 | Apremilast | 0.3 |
| 2 | Dimethyl isosorbide | 5 |
| 3 | Transcutol | 4 |
| 4 | PEG 540 Blend | 83.7 |
| 5 | Polysorbate 60 | 2 |
| 6 | PEG 6000 | 5 |
| | Total | 100 |

Topical Ointment formulation was prepared according to the process described in Example 4.

### Example 7 (Apremilast Cream)

TABLE 7

| Sr. | | 7a | 7b | 7c | 7d |
|---|---|---|---|---|---|
| no | Ingredients | % (w/w) | % (w/w) | % (w/w) | % (w/w) |
| 1 | Apremilast | 0.30 | 0.3 | 0.3 | 0.3 |
| 2 | Light Mineral oil | 5.00 | 6.0 | 5.0 | 5.0 |
| 3 | Glyceryl stearate | 3.00 | 4.0 | 3.0 | 3.0 |
| 4 | Cetyl alcohol | 4 | 7.0 | 5.0 | 3.0 |
| 5 | Stearyl Alcohol | 3.50 | — | 3.0 | 3.0 |
| 6 | Sorbitan mono- | 2.50 | — | 0.5 | 0.5 |
| | stearate | | | | |
| 7 | Phenoxyethanol | 1 | — | — | — |
| 8 | Stearic acid | — | — | — | 3.0 |
| 9 | Transcutol | 5 | 5.0 | 5.0 | 5.0 |
| 10 | Sodium edetate | 0.10 | — | — | — |
| 11 | Sodium Lauryl | 0.50 | — | — | — |
| | sulphate (SLS) | | | | |
| 12 | Dimethyl-isosorbide | 5 | 5.0 | 5 | 5.0 |
| 13 | Polysorbate 60 | — | 2.0 | 4.0 | 4.5 |
| 14 | Methyl paraben | — | 0.7 | 0.7 | 0.1 |
| 15 | Propyl paraben | — | 0.3 | 0.3 | 0.03 |
| 16 | Ceteareth-20 | — | — | 4.0 | 4.0 |
| 17 | Water | Q.s to 100 | Q.s to 100 | Q.s to 100 | Q.s to 100 |

### Process for the Preparation of Apremilast Cream

Cetyl alcohol, Ceteareth-20, stearyl alcohol, stearic acid and glyceryl stearate (As per table 7) were melted and mixed with light mineral oil. Sorbitan monostearate was melted and mixed with the prepared solution. Further, transcutol, methyl and propyl paraben or phenoxyethanol, SLS, sodium edetate (As per table 7) were added to prepare oil phase. Polysorbate 60 was added to water and melted to obtain aqueous solution. The aqueous solution was added to the oil phase and homogenized. Drug solution was prepared by dissolving apremilast in dimethyl isosorbide. The drug solution was added to the oil phase to obtain cream formulation.

### Example 8 (Apremilast Gel)

TABLE 8

| Sr. | | Example 8 |
|---|---|---|
| no | Ingredients | % (w/w) |
| 1 | Apremilast | 0.3 |
| 2 | Transcutol | 5.0 |
| 3 | Carbopol ® 980 | 0.2 |
| 4 | Methyl Paraben | 0.2 |
| 5 | Water | Q.s to 100% |
| 6 | Dimethyl isosorbide | 1.0 |
| 7 | Sodium Hydroxide | q.s. |
| | Observation | Flowable gel |

### Process for the Preparation of Apremilast Gel

Methyl paraben was dissolved in water, Carbopol 980 was added to it and dissolved. Drug phase was prepared by dissolving Apremilast in Dimethyl isosorbide and transcutol. Drug phase was added slowly to the polymer solution and pH was adjusted to pH 6.0 to 6.5 with sodium hydroxide.

### Example 9

### In-Vivo Efficacy Studies

Mouse model for FITC (Fluorescein isothiocyanate)-induced allergic cutaneous inflammation holds resemblance with human acute atopic dermatitis lesions. Sensitization of mice by topical application of FITC results in increased IgE levels, as well as the development of FITC specific Th2 cells. Antigen challenge by applying FITC to the mouse ear 6 days post-sensitization results in cutaneous inflammation characterized by edema and large infiltration of inflammatory cells as mononuclear cells, eosinophils and neutrophils. (Boehme S A et al., Int Immunol. 2009 January; 21(1):81-93).

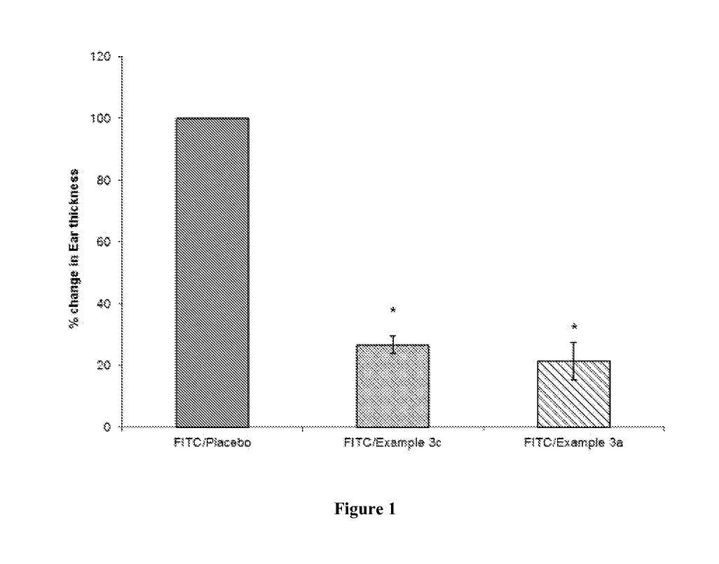

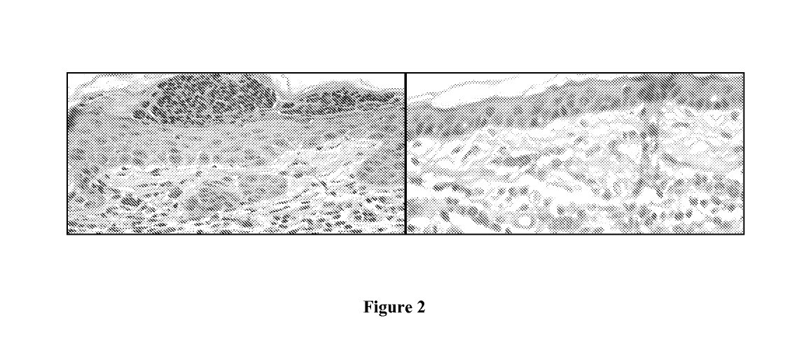

Efficacy of apremilast by topical formulation application was evaluated in FITC-induced allergic cutaneous inflammation in female BALB/c mice. Mice were prepared for sensitization on day 0 by carefully removing hair from their ventral skin using an electric hair clipper under isoflurane anesthesia. On day 1 and day 2, 400 μL of 0.5% FITC solution/Vehicle was painted on the shaved ventral skin (dissolved in acetone:dibutyl phthalate, 1:1, v/v). Further on day 8, the mice were challenged with 0.5% FITC/Vehicle on the right ear (20 μl volume). Mice in FITC challenged groups were treated with 30 μL formulation prepared according to example 3c and 3a or placebo, on right ear pinna (15 μL on inner side of right pinna and 15 μL on outer side of right pinna) applied topically on right ear 4 hours before and 4 hours after the FITC challenge. Mice in vehicle group were applied placebo formulation. 24 hours after the FITC challenge, the right ear thickness was determined using digital vernier caliper in anesthetized mice and histopathological analysis of formalin fixed right ear sections was performed after Hematoxylin and eosin staining. Results are shown in FIG. 1 (Data represent mean±standard error of mean. * denotes p<0.05 versus FITC/placebo group using oneway ANOVA followed by Dunnett's post hoc analysis.) and FIG. 2.

### Observation:

% change in ear thickness in mice treated with apremilast topical formulation of present invention was very less (less than around 30%) as compared to that in mice treated with placebo formulation and attenuation in the inflammatory cell infiltration was observed in mice treated with apremilast topical formulation of present invention as compared to placebo.

### Example 10

### Pharmacokinetics of Apremilast in Female Balb C Mice

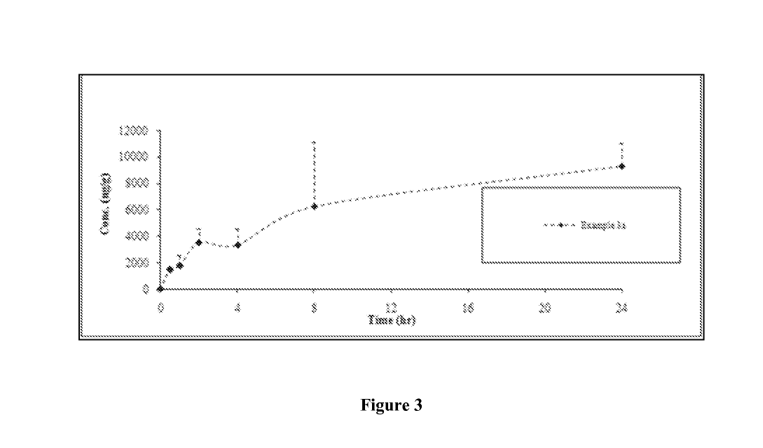

Pharmacokinetics of Apremilast was studied in Female Balb C Mice by topical route with solution of Example 3a (^{˜}42 mg/kg in Vol. 30 μL). In the study, total 18 animals were included and dermis was collected from three animals at each six different time points i.e. 0.5, 1, 2, 4, 8 and 24 hrs post administration. Dermis concentrations of Apremilast was quantified by LC/MS-MS method & depicted in FIG. 3 along with PK parameters in the Table 9.

Dermis concentrations of Apremilast was quantified by LC/MS-MS method.

TABLE 9

| Parameter | Example 3a |
|---|---|
| Cₘₐₓ (ng/gm) | 9306.64 |
| Cₗₐₛₜ (ng/gm) | 9306.64 |
| AUCₗₐₛₜ (hr*ng/gm) | 154063.46 |

1. A topical pharmaceutical composition comprising apremilast in an amount of about 0.1 to 5% w/w of the total composition and one or more pharmaceutical excipient(s).

2. The topical pharmaceutical composition according to claim 1, wherein the composition is in the form of cream, ointment, gel, transdermal formulations, foam, spray, lotion, solution, emulsion or suspension.

3. The topical pharmaceutical composition according to claim 1 wherein the pharmaceutical excipient is a solubilizer or a penetration enhancer or mixtures thereof.

4. The topical pharmaceutical composition according to claim 3, wherein the apremilast and the solubilizer are present in ratio of about 1:1 to 1:50, preferably in ratio of about 1:5 to 1:50.

5. The topical pharmaceutical composition according to claim 3, wherein the solubilizer and the penetration enhancer are present in a ratio of about 1:0.1 to 1:5.

6. The topical pharmaceutical composition according to claim 3, wherein the solubilizer is selected from dimethyl malonate, diethyl succinate, diethyl glutarate, diethyl adipate, dipropyl adipate, dibutyl sebacate, diisopropyl sebacate, diethyl pimelate, diethyl suberate, diethyl azelate, dibutyl adipate, dibutyl sebacate, methyl ethyl succinate, diethyl ethyl-isopropylmalonate, diethyl isosuccinate, benzyl alcohol, benzyl benzoate, cyclodextrin, glycerine monostearate, lecithin, butylene glycol, dibutyl phthalate, diethyl phthalate, dimethyl ether, diethyl ether, ethyl acetate, ethyl lactate, ethyl oleate, glycofurol, isopropyl alcohol, triacetin, triethanolamine, hexylene glycol, oils, dimethyl sulfoxide and dimethyl isosorbide.

7. The topical pharmaceutical composition according to claim 6, wherein the solubilizer is dimethyl isosorbide.

8. The topical pharmaceutical composition according to claim 3, wherein the penetration enhancer is selected from monooleate of ethoxylated glycerides with 8 to 10 ethylene oxide units, polyethylene glycol, Alpha-Tocopherol Polyethylene glycol succinate, diethylene glycol monoethyl ether, tetrahydrofurfuryl alcohol PEG ether, s dimethylsulphoxide, dodecyl methyl sulfoxide, 1-Butyl-2-azacycloheptanones, 1-Hexyl-2-azacycloheptanones, 1-Octyl-2-azacycloheptanones, 1-dodecylazacycloheptan-2-one, 1-ethyl-2-pyrrolidone, 1-butyl-2-pyrrolidones, 1-Hexyl-2-pyrrolidinone, 1-Octyl-2-pyrrolidinone, 1-Decyl-2-pyrrolidinone, 1-Dodecyl-2-pyrrolidinone, N-methyl-2-pyrrolidone, 2-pyrrolidone, 2-(1-Butyl)-2-methyl-1,3 dioxolane, 2-(1-Hexyl)-2-methyl-1,3 dioxolane and 2 N-nonyl-1,3-dioxolanes.

9. The topical pharmaceutical composition according to claim 8, wherein the penetration enhancer is diethylene glycol monoethyl ether.

10. The topical pharmaceutical composition according to claim 1, wherein the composition is an aqueous composition.

11. A process of preparation of topical composition comprising apremilast and one or more pharmaceutical excipients comprising:
a) preparing a solution of 0.1-5% of apremilast in one or more pharmaceutical excipients, preferably solubilizers;
b) optionally mixing the solution of step a) with one or more pharmaceutically acceptable excipient(s);
c) preparing a topical pharmaceutical composition in the form of cream, ointment, gel, transdermal formulations, foam, spray, lotion, solution, emulsion or suspension.
